(19) United States
(12) Patent Application Publication
(10) Pub. No.: US 2025/0362365 A1
(43) Pub. Date: Nov. 27, 2025

(54) LOW FIELD MAGNETIC RESONANCE IMAGING STROKE IDENTIFICATION SEQUENCE AND DEVICE FOR IDENTIFYING THE LOW FIELD MAGNETIC RESONANCE IMAGING STROKE IDENTIFICATION SEQUENCE

(71) Applicants: Beijing Tiantan Hospital, Capital Medical University, Beijing (CN); Ray Plus Medical Technology Co., Ltd., Beijing (CN)

(72) Inventors: Yongjun Wang, Beijing (CN); Jing Jing, Beijing (CN); Ning Wang, Beijing (CN); Yihuai Wang, Beijing (CN); Xuewei Xie, Beijing (CN); Bingshan Xue, Beijing (CN); Chunguang Zhang, Beijing (CN)

(21) Appl. No.: 19/202,269

(22) Filed: May 8, 2025

Related U.S. Application Data
(63) Continuation of application No. PCT/CN2025/083747, filed on Mar. 20, 2025.

(30) Foreign Application Priority Data
May 21, 2024 (CN) 2024106314576

(51) Int. Cl. G01R 33/561; A61B 5/00; A61B 5/055; G01R 33/56

(52) U.S. Cl. CPC G01R 33/5617; A61B 5/0042; A61B 5/055; G01R 33/5602; G01R 33/5608

(57) ABSTRACT

A low field magnetic resonance imaging (MRI) stroke identification sequence uses an inversion recovery sequence. TE is set to the minimum or near-minimum value achievable by a system to minimize an impact of T2 effect on a signal S. Additionally, it selects combinations of TR values and corresponding TI values to make signal from cerebral hemorrhage appear as high signal, and signals from cerebral infarct tissue and cerebral parenchyma appear as isointense or low signal. This allows for rapid and accurate determination of hemorrhagic stroke and, by utilizing low-field magnetic resonance, enhances the accessibility for patients.

### CROSS-REFERENCE TO RELATED APPLICATIONS

This application is a continuation of International Application No. PCT/CN2025/083747, filed on Mar. 20, 2025. The international Application claims priority to Chinese patent application No. CN 202410631457.6, filed to China National Intellectual Property Administration (CNIPA) on May 21, 2024, which is herein incorporated by reference in its entirety.

### TECHNICAL FIELD

The disclosure relates to the field of magnetic resonance imaging (MRI) technologies, and particularly to a low field MRI stroke identification sequence and a device for identifying the low field MRI stroke identification sequence.

### BACKGROUND

Stroke, also known as cerebral apoplexy, is an acute cerebrovascular disease that includes hemorrhagic stroke and ischemic stroke. The hemorrhagic stroke is also referred to as cerebral hemorrhage, and the ischemic stroke is also known as cerebral ischemia or cerebral infarction. A time for rescuing patients with the cerebral infarction is very limited. It is generally believed that an optimal treatment time for the stroke is within 4.5 hours (h), and exceeding 6 h often means missing the best opportunity for rescue. Therefore, in the diagnosis and treatment of the stroke, determining whether the patient has the cerebral hemorrhage, the cerebral infarction, or infarction with hemorrhage is the key for doctors to decide on a therapeutic regimen. How to quickly and accurately assess the patient's condition is crucial to the treatment outcome and the patient's life.

Among the existing diagnostic auxiliary techniques and procedures, non-contrast computed tomography (CT) of the head is the primary diagnostic method used in hospitals for the cerebral hemorrhage. The CT is highly sensitive to the cerebral hemorrhage and is considered the “gold standard” for diagnosing the cerebral hemorrhage. It is a mandatory examination for patients with suspected stroke and serves as an important imaging basis in the current stroke diagnosis and treatment process. However, the CT is not sensitive to acute ischemic stroke, with inconspicuous lesion images. It is generally believed that the CT can only detect the cerebral infarction lesions larger than 24 hours, making it difficult to identify earlier infarctions and prone to misdiagnosing early cerebral infarction.

Some hospitals also use a dedicated superconducting MRI device to differentiate and examine stroke, which can improve the detection rate of acute cerebral infarction and accurately determine a time window of the cerebral infarction. The MRI can also enhance a detection rate of the cerebral hemorrhage, especially for hemorrhagic transformation after the cerebral infarction and for hemorrhages that have occurred over a longer period of time. However, currently, the superconducting MRI device belongs to high-field strength devices, with commonly used field strength of 1.5 Tesla (T) and 3.0 T. The devices need to be installed in specialized electromagnetically shielded rooms, which is extremely inconvenient for patients with stroke where every second counts in rescue efforts and can easily delay the opportunity for timely treatment. The patients with metal implants or other medical assistive devices in their bodies cannot undergo MRI scans. During MRI scanning, metal objects absorb electromagnetic waves, which are converted into heat. The higher the magnetic field strength, the more heat is generated. Under the influence of a strong magnetic field, the heat generated can burn the patients. Therefore, such patients are not eligible for the M RI, reducing its accessibility.

### SUMMARY

To solve above technical problems, the disclosure provides a low field MRI stroke identification sequence with high accessibility and the ability to quickly and accurately determine hemorrhagic stroke, and a device for identifying the low field MRI stroke identification sequence.

The low field MRI stroke identification sequence uses an inversion recovery sequence, and a formula of a relative signal intensity of the inversion recovery is expressed as follows:

$$S = {\sum }_{i = 1}^{N} P {D}_{i} ( 1 - 2 exp ( \frac{TI}{T {1}_{i}} ) + exp ( - \frac{TR - {TE}_{last}}{T {1}_{i}} ) ) \times exp ( - \frac{TE}{T {2}_{i}} )$$

where S represents the relative signal intensity; PD represents a proton density of tissue; TI represents an inversion recovery time of the inversion recovery sequence; T1 represents a T1 relaxation time of the tissue; TR represents a repetition time of the inversion recovery sequence; TEₗₐₛₜ represents a last echo time in a multi-echo sequence; TE represents an effective echo time; T2 represents a T2 relaxation time of the tissue; i=1, 2, . . . , N, N represents N types of components, cerebral parenchyma and cerebral hemorrhage are modeled as a single-component model with N=1, cerebral infarction tissue is modeled as a two-component model with N =2, and a subscript i represents a value of an i-th type of tissue.

The TE is set to a minimum value or a near-minimum value achievable by a system, thereby making

$$exp ( - \frac{T E}{T {2}_{i}} )$$

as close to 1 as possible, and minimizing an influence of an T2 effect on the relative signal intensity S.

A combination of a combination of a TR value and a corresponding TI value is selected, thereby making a signal from the cerebral hemorrhage as high signal, and a signal from the cerebral infarction tissue and the cerebral parenchyma as an isointense or a low signal.

In an embodiment, selection of the combination of the TR value and the corresponding TI value conforms to a principle of a fastest clinical scanning speed. In MRI scans, the TR is usually longer than the time needed for all essential activities. This is because a certain waiting time is required for the signal to relax sufficiently between two excitation pulses. To boost acquisition efficiency, other slice information is acquired during the waiting time. This technique is called multi-slice acquisition. It can be seen that the principle of the fastest clinical scanning speed is to completely occupy a TR value with data collected from different levels, without any waiting time. For example, when the TR value is 1000 milliseconds (ms) during the scanning, each layer takes 100 ms to complete the necessary excitation, encoding, and acquisition for each slice, that is to say, the TR value of 1000 ms for scanning 10 layers is the most efficient, as it reduces waiting time to zero. If 11 layers are required clinically, the 11 layers can't fit into a single TR period of 1000 ms. The scan must be split into two groups, leading to longer waiting times within each group. However, with a TR of 1100 ms, which doesn't affect contrast, the scanning of the 11 layers becomes the most efficient. In this case, a corresponding relationship between the TR value and the corresponding TI value must conform to a fitting result to satisfy clinical contrast requirements.

In an embodiment, a corresponding relationship between the TR value and the corresponding TI value conforms to a fitting result as follows:

$$TR = 0.0006925 \times {TI}^{2} + 0.74 \; 26 \times TI + 67.78 .$$

In an embodiment, the corresponding TI value is a fixed value, and the TR value fluctuates up and down by 10% according to the fitting result; or the TR value is a fixed value, and the corresponding TI value fluctuates up and down by 10% according to the fitting result.

In an embodiment, a strength of the low field is 0.23 Tesla (T), a TE value is 24 ms, the TR value is 900 ms, and the corresponding TI value is 685 ms.

In an embodiment, a strength of the low field is 0.23 Tesla (T), a TE value is 24 ms, the TR value is 1100 ms, and the corresponding TI value is 800 ms.

In an embodiment, a strength of the low field is 0.23 T, a TE value is 24 ms, the TR value is 1500 ms, and the corresponding TI value is 1000 ms.

In an embodiment, a PD value is obtained by measuring a signal intensity of a proton weighted image.

In an embodiment, before using a fast spin echo sequence, inversion recovery pulses with a series of different TI values are applied to the relative signal intensity formula, then a signal size of a region of interest (ROI) is related to a T1 value. A T1 measurement value is obtained by fitting using a formula expressed as follows:

$$S ( τ ) = α \times ( 1 - 2 exp ( - \frac{τ}{T 1} ) + exp ( - \frac{TR - {TE}_{last}}{T 1} ) )$$

where S(τ) represents a signal intensity of the tissue; α represents a weight coefficient; and τ represents a series of different TI values (i.e., the T1 value of a scanning sequence).

In an embodiment, other parameters are kept unchanged except for a TE value, then a fast spin echo sequence is used to obtain a T2 measurement value based on a correlation between the TE and the T2 by using a fast spin echo sequence. The other parameters include TR value and so on. The T2 measurement value is obtained by fitting using a formula expressed as follows:

$$S ( β ) = α \times exp ( - \frac{β}{T 2} )$$

where S(β) represents a signal intensity of the tissue; a represents a weight coefficient; and β represents a series of echo times.

To solve above technical problems, the disclosure provides a device for identifying the low field MRI stroke identification sequence, which includes a magnetic resonance scanner, and the magnetic resonance scanner is configured to identify the low field MRI stroke identification sequence mentioned above.

Compared to the related art, the disclosure adopts a inversion recovery, a system that can achieve the a minimum value or a near-minimum value of TE is adopted, which can minimize an influence of a T2 effect on the relative signal intensity S. Simultaneously, the selection of a combination of a TR value and a TI value, which makes a signal from the cerebral hemorrhage as a high signal and a signal from the cerebral infarction tissue and the cerebral parenchyma as an isointense or a low signal, thus enabling rapid and accurate diagnosis of hemorrhagic stroke. In addition, a low field magnetic resonance imaging is also used to improve the accessibility for patients.

### BRIEF DESCRIPTION OF DRAWINGS

In order to provide a clearer explanation of the embodiments of the disclosure or the technical solutions in the related art, a brief introduction will be given to the attached drawings required for the description of the embodiments or the related art.

FIG. 1A illustrates a T2-weighted imaging (T2) image of a pig cerebral hemorrhage experiment started at 0^{th} hour (h).

FIG. 1B illustrates a fluid attenuated inversion recovery (FLAIR) image of the pig cerebral hemorrhage experiment started at 0^{th} h.

FIG. 1C illustrates a diffusion weighted imaging (DWI) image of the pig cerebral hemorrhage experiment started at 0^{th} h.

FIG. 1D illustrates a T1-weighted imaging (T1) image of the pig cerebral hemorrhage experiment started at 0^{th} h.

FIG. 2A illustrates a T2 image of the pig cerebral hemorrhage experiment ended at 17^{th} h.

FIG. 2B illustrates a FLAIR image of the pig cerebral hemorrhage experiment ended at 17^{th} h.

FIG. 2C illustrates a DWI image of the pig cerebral hemorrhage experiment ended at 17^{th} h.

FIG. 2D illustrates a T1 image of the pig cerebral hemorrhage experiment ended at 17^{th} h.

FIG. 3A illustrates a T1 relaxation time curve of the pig cerebral hemorrhage experiment.

FIG. 3B illustrates a T2 relaxation time curve of the pig cerebral hemorrhage experiment.

FIG. 3C illustrates a proton density (PD) normalization curve of the pig cerebral hemorrhage experiment.

FIG. 3D illustrates a T1 weighted curve of the pig cerebral hemorrhage experiment.

FIG. 3E illustrates a T2 weighted curve of the pig cerebral hemorrhage experiment.

FIG. 3F illustrates a FLAIR curve of the pig cerebral hemorrhage experiment.

FIG. 4A illustrates a T2 image of a pig cerebral ischemia experiment started at 0^{th} h.

FIG. 4B illustrates a FLAIR image of the pig cerebral ischemia experiment started at 0^{th} h.

FIG. 4C illustrates a DWI image of the pig cerebral ischemia experiment started at 0^{th} h.

FIG. 4D illustrates a T1 image of the pig cerebral ischemia experiment started at 0^{th} h.

FIG. 5A illustrates a T2 image of the pig cerebral ischemia experiment ended at 24^{th} h.

FIG. 5B illustrates a FLAIR image of the pig cerebral ischemia experiment ended at 17^{th} h.

FIG. 5C illustrates a DWI image of the pig cerebral ischemia experiment ended at 24^{th} h.

FIG. 5D illustrates a T1 image of the pig cerebral ischemia experiment ended at 24^{th} h.

FIG. 6A illustrates a T1 relaxation time curve of the pig cerebral ischemia experiment.

FIG. 6B illustrates a T2 relaxation time curve of the pig cerebral ischemia experiment.

FIG. 6C illustrates a PD normalization curve of the pig cerebral ischemia experiment.

FIG. 6D illustrates a T1 weighted curve of the pig cerebral ischemia experiment.

FIG. 6E illustrates a T2 weighted curve of the pig cerebral ischemia experiment.

FIG. 6F illustrates a FLAIR curve of the pig cerebral ischemia experiment.

FIG. 7A illustrates a fitting diagram with an effective echo time (TE) of 24 milliseconds (ms) and a repetition time (TR) of 900 ms.

FIG. 7B illustrates a fitting diagram with a TE of 24 ms and a TR of 1100 ms.

FIG. 7C illustrates a fitting diagram with a TE of 24 ms and a TR of 1500 ms.

FIG. 8 illustrates a relationship curve of TE and TR (TR/TI) with a unit of ms.

FIG. 9A illustrates a hemorrhage image with TR/TI=900/685 ms, where a thick line circle represents a hemorrhage area, and a thin line circle represents a cerebral parenchyma area.

FIG. 9B illustrates a hemorrhage image with TR/TI=1100/800 ms, where a thick line circle represents a hemorrhage area, and a thin line circle represents a cerebral parenchyma area.

FIG. 9C illustrates a hemorrhage image with TR/TI=1500/1000 ms, where a thick line circle represents a hemorrhage area, and a thin line circle represents a cerebral parenchyma area.

FIG. 10A illustrates a DWI image of a brain in a first case.

FIG. 10B illustrates an apparent diffusion coefficient (ADC) image of the brain in the first case.

FIG. 10C illustrates a FLAIR image of the brain in the first case.

FIG. 10D illustrates a PD image of the brain in the first case.

FIG. 11A illustrates a DWI image of a brain in a second case.

FIG. 11B illustrates an A DC image of the brain in the second case.

FIG. 11C illustrates a FLAIR image of the brain in the second case.

FIG. 11D illustrates a PD image of the brain in the second case.

FIG. 11E illustrates a typical image combination of DWI, ADC, FLAIR, and PD images of the brain in the second case.

FIG. 12A illustrates a DWI image of a brain in a third case.

FIG. 12B illustrates an A DC image of the brain in the third case.

FIG. 12C illustrates a FLAIR image of the brain in the third case.

FIG. 12D illustrates a PD image of the brain in the third case.

FIG. 13A illustrates a DWI image of a brain in a fourth case.

FIG. 13B illustrates an ADC image of the brain in the fourth case.

FIG. 13C illustrates a FLAIR image of the brain in the fourth case.

FIG. 13D illustrates a PD image of the brain in the fourth case.

FIG. 14A illustrates a DWI image of a brain in a fifth case.

FIG. 14B illustrates an ADC image of the brain in the fifth case.

FIG. 14C illustrates a FLAIR image of the brain in the fifth case.

FIG. 14D illustrates a PD image of the brain in the fifth case.

FIG. 15A illustrates a DWI image of a brain in a sixth case.

FIG. 15B illustrates an ADC image of the brain in the sixth case.

FIG. 15C illustrates a FLAIR image of the brain in the sixth case.

FIG. 15D illustrates a PD image of the brain in the sixth case.

In the above case images, circles indicate that highlighted signal areas are the lesion areas.

DWI image: A type of MRI sequence in clinical diagnostic imaging, where high signal intensity on the images is generally considered to be caused by pathological abnormalities.

ADC image: A type of MRI sequence in clinical diagnostic imaging, which is a calculated value image derived from the DWI image. By combining the signal intensity of the corresponding lesion areas in both ADC image and DWI image for a comprehensive diagnosis, it is possible to determine whether the lesion is an acute stroke.

FLAIR image: A type of MRI sequence image in clinical diagnostic imaging, in which other tissues maintain a T2-weighted appearance while cerebrospinal fluid appears as low signal intensity. Bright areas in the image are generally considered to be caused by pathological changes.

PD image: A type of MRI sequence in clinical diagnostic imaging, which is primarily used to determine whether a suspected stroke lesion is due to cerebral ischemia or cerebral hemorrhage.

### DETAILED DESCRIPTION OF EMBODIMENTS

The preferred embodiments of the disclosure will be described in detail with reference to the attached drawings.

### I. Magnetic Field Strength B₀

Magnetic resonance imaging (MRI) market is overwhelmingly dominated by device with high-field systems, especially for medical or clinical MRI applications. A general trend in medical imaging is to produce MRI scanners with increasingly higher field strengths, where a vast majority of clinical MRI scanners operate at 1.5 Tesla (T) or 3 T, and even higher field strengths such as 7 T and 9 T are used in research environments. “High field” generally refers to MRI systems currently used in clinical settings, more specifically, the MRI systems that operate with a main magnetic field (i.e., B₀ field) of 1.0 T or above. Clinical systems that operate between 0.5 T and 1.0 T are also commonly described as “mid-field.” Field strengths between approximately 0.3 T and 0.5 T are characterized as “mid-low field.” In contrast, “low field” typically refers to the MRI systems that operate with a B₀ field within a range of approximately 0.18 T to 0.3 T. Low-field MRI systems that operate with a B₀ field less than 0.18 T are referred to as “ultra-low field.”

### II. MRI Manifestations of Cerebral Hemorrhage and Cerebral Ischemia (Cerebral Infarction)

Some studies have shown that the magnetic resonance characteristics of cerebral hemorrhage and cerebral ischemia are usually complex and variable. These manifestations are not only time-dependent but also highly related to the magnetic field strength.

For cerebral hemorrhage in a super acute phase (within 6 h), the increase in paramagnetic substances such as deoxyhemoglobin within red blood cells (erythrocytes) leads to a local reduction in the T2* effect. Therefore, in high-field magnetic resonance imaging, it usually presents characteristics such as isointense or low signal intensity. Since the susceptibility effect is positively correlated with the square of the magnetic field strength, in the low-field magnetic resonance imaging, super acute phase hemorrhage is usually less affected by this, presenting as persistent high signal on DWI and FLAIR.

For the cerebral ischemia in the super acute phase, the magnetic resonance imaging typically shows high signal intensity on DWI and isointense signal on FLAIR. As time progresses, due to damage to the blood-brain barrier and other factors, the FLAIR signal in the cerebral infarction area will gradually become high.

It can be seen that in the low-field magnetic resonance imaging, both cerebral hemorrhage and cerebral ischemia may exist at certain times when both DWI and FLAIR show high signals simultaneously, making it impossible to distinguish between them. Therefore, the following animal experiments are conducted to find a low field MRI stroke identification sequence that can quickly and accurately determine hemorrhagic stroke.

### 1. Quantitative Analysis Method for T1 and T2

T1 Measurement value of the tissue: Before using a fast spin echo sequence, a series of TI values is applied to the relative signal intensity formula, then a signal size of a region of interest (ROI) is related to a TI value. A T1 measurement value is obtained by fitting using a formula expressed as follows:

$$S ( τ ) = α \times ( 1 - 2 exp ( - \frac{τ}{T 1} ) + exp ( - \frac{TR - {TE}_{last}}{T 1} ) )$$

where S(τ) represents a signal intensity of the tissue; a represents a weight coefficient; τ represents TI values of a scanning sequence; TR represents the repetition time of the inversion recovery; TEₗₐₛₜ represents the last echo time in the multi-echo sequence; and T1 represents the T1 value of the tissue.

Fixed scanning parameters for this sequence are as follows: In a first round of scanning, the repetition time TR is 4000 ms, and the last echo time TE is 107 ms. A series of TI values, denoted as t, are set to [100 150 200 250 400 600 800 1000] ms. In a second round of scanning, the repetition time TR is 10000 ms, and the last echo time TE remains at 107 ms. The series of TI values, i.e., t, are set to [3500 4000 4500] ms. A series of signal intensity values S(τ) corresponding to the TI values are obtained. By numerically fitting the data of τ and S(τ) using the above formula, the T1 measurement value is finally derived.

### T2 Measurement Value

Other parameters are kept unchanged except for the TE, then a T2 measurement value is obtained based on a correlation between the TE and the T2 by using a fast spin echo sequence. The T2 measurement value is obtained by fitting using a formula expressed as follows:

$$S ( β ) = α \times exp ( - \frac{β}{T 2} )$$

where S(β) represents a signal intensity of the tissue; α represents a weight coefficient; β represents a series of echo times; and T2 represents T2 value of the tissue.

The scanning parameters for this sequence are as follows: TR is 4000 ms, and a series of echo times β are set to [42, 56, 70, 84, 98, 112] ms. A series of S(β) values corresponding to the series of echo times β are obtained. By numerically fitting the data of β and S(β) using the above formula, the T2 measurement value is finally derived.

PD value measurement: According to the principles of magnetic resonance, the disclosure directly uses the signal intensity of the PD-weighted image to calculate the relative PD values between tissues. This value represents the signal value of the PD-weighted image, not the absolute PD of the tissue. Since the signal value of the PD-weighted image is extremely large, the disclosure normalizes its magnitude to a single-digit level in the illustration, which does not affect the expression of trends in the illustration. The scanning parameters for this sequence are: TE is the minimum TE of the system, and TR is 10000 ms.

Selection of ROI: For the estimation of T1 values and T2 values of the cerebral parenchyma, the basal ganglia region is chosen. This area (ROI) has relatively uniform signals, is less affected by cerebrospinal fluid and other signals, and has a relatively inconspicuous volume effect, which can reflect the signal characteristics of the cerebral parenchyma.

### 2. Experimental Equipment

The equipment used in the disclosure is the mobile head and neck magnetic resonance system ACUTA Elfin manufactured by Ray Plus Medical Technology Co., Ltd. The basic parameters are as follows: a nominal B₀ value: 0.23 T±0.01 T, a maximum spatial encoding gradient: 25 milli Tesla per meter (mT/m), a maximum gradient switching rate: 60 tesla per meter per second (T/m/s).

### 3. Hemorrhage Experimental Results

In the experiment, pig venous blood is collected and injected into a basal ganglia region of the pig's brain. The MRI clearly shows a hemorrhagic focus. Starting immediately after the blood injection, one round of MRI sequence scanning is completed every hour to continuously assess the signal changes of the hemorrhagic focus over a period of 17 hours.

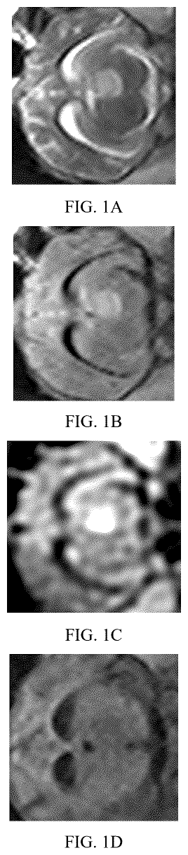

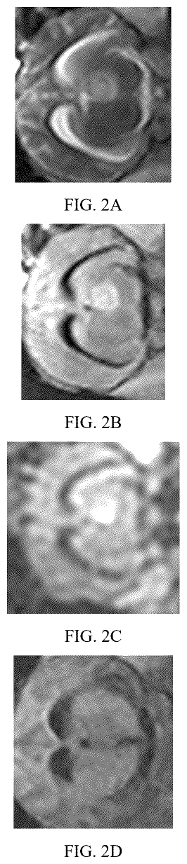

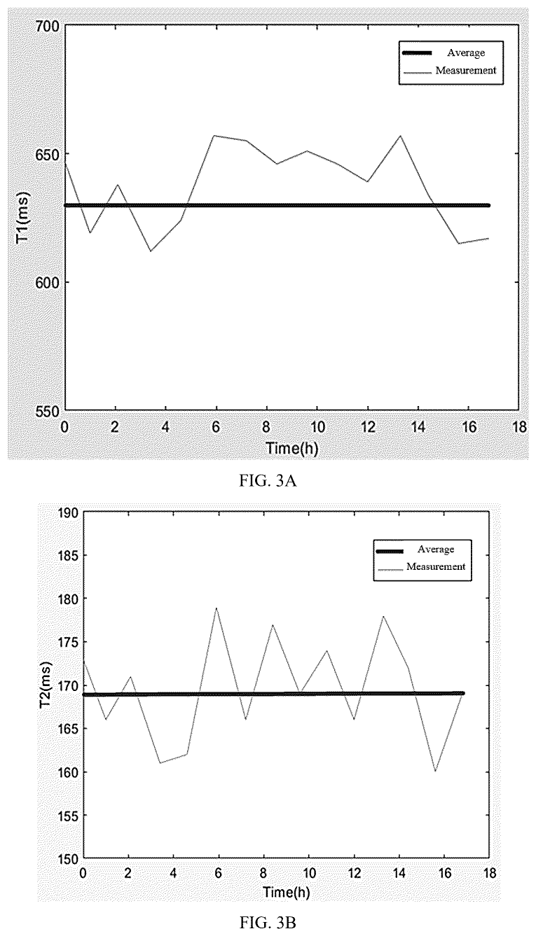

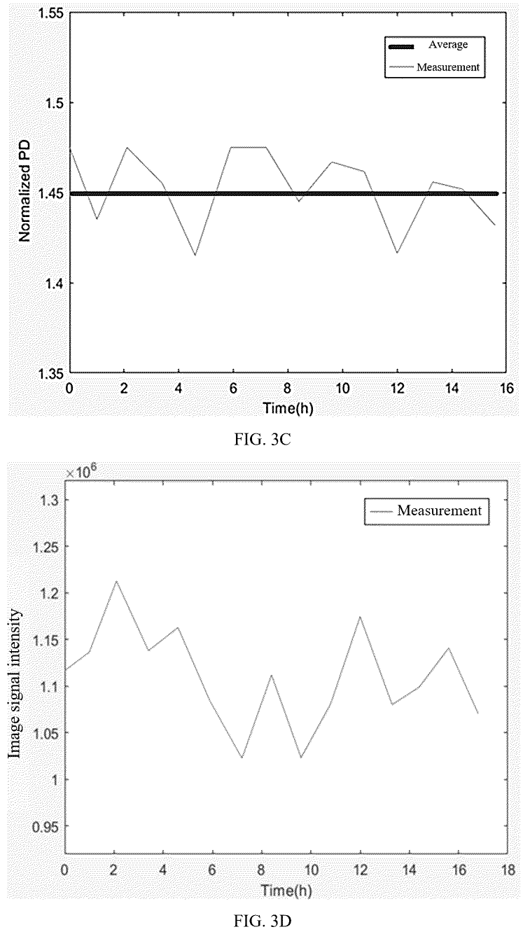

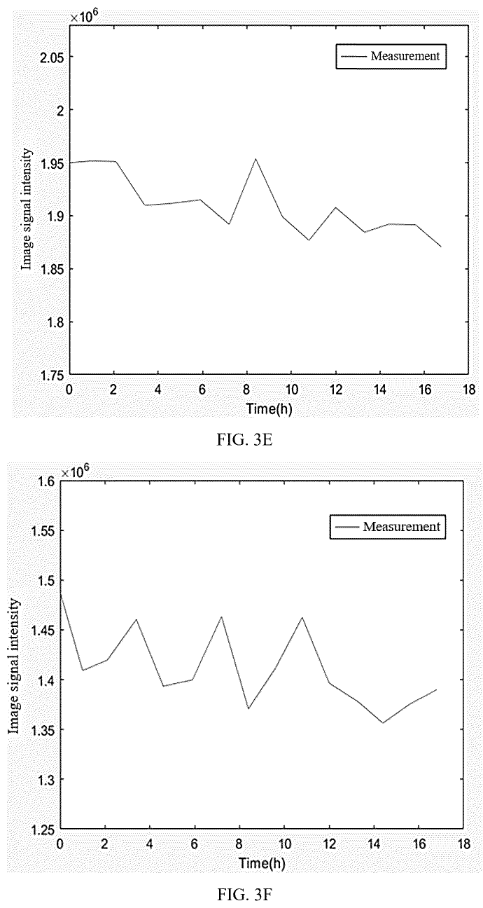

Refer to FIGS. 1A-3F. Using the above measurement method, measurements are continuously calculated hourly, and it is found that there is no significant change within 17 hours. Therefore, the data from the 17 hours are averaged to obtain a mean value for convenience in subsequent calculations. By directly measuring the signal intensity of T1-weighted (T1WI) images, T2-weighted (T2WI) images, FLAIR images, it is observed that under 0.23 T MRI, the hemorrhagic focus shows slightly low signal on the TIWI images, and high signal on the T2WI images, FLAIR images, and DWI images within 17 hours, with no significant change in signal intensity over time.

Based on the above measurement method, the average values of T1, T2, and PD for both cerebral parenchyma and hemorrhage are as follows:

TABLE 1

| | Brain | Cerebral |
|---|---|---|
| | Parenchyma | Hemorrhage |
| T1 (ms) | 420 | 630 |
| T2 (ms) | 105 | 169 |
| PD | 1.15 | 1.45 |
| (normalization) | | |

### 4. Ischemia Experiment Results

In this experiment, the analyzed area of cerebral ischemia is located in the medulla oblongata. The cerebral ischemia model also starts from the formation of an infarct focus and continuously assessed the signal changes within the infarct area over a period of 24 hours.

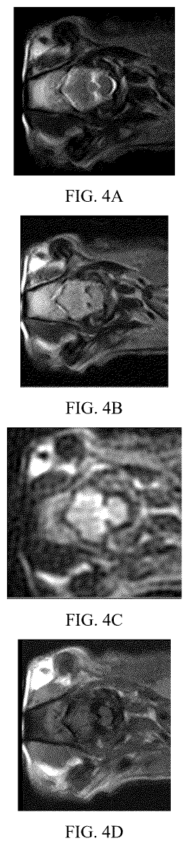

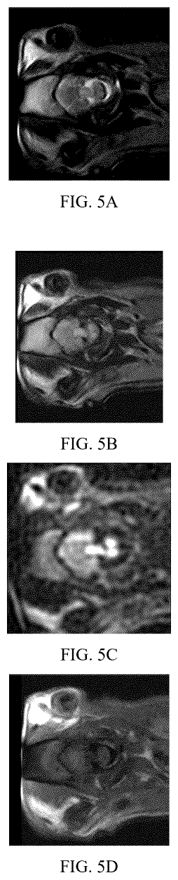

Refer to FIGS. 4A-6F. Through 24-hour monitoring, it is found that under 0.23 T magnetic resonance, the T2WI signals, FLAIR signals, and DWI signals of cerebral ischemia continued to increase, while the T1WI signals continued to decrease.

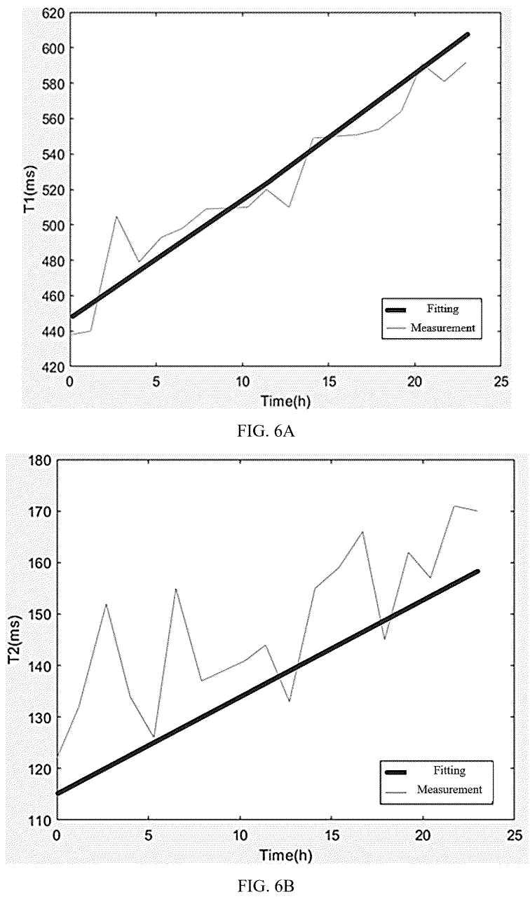

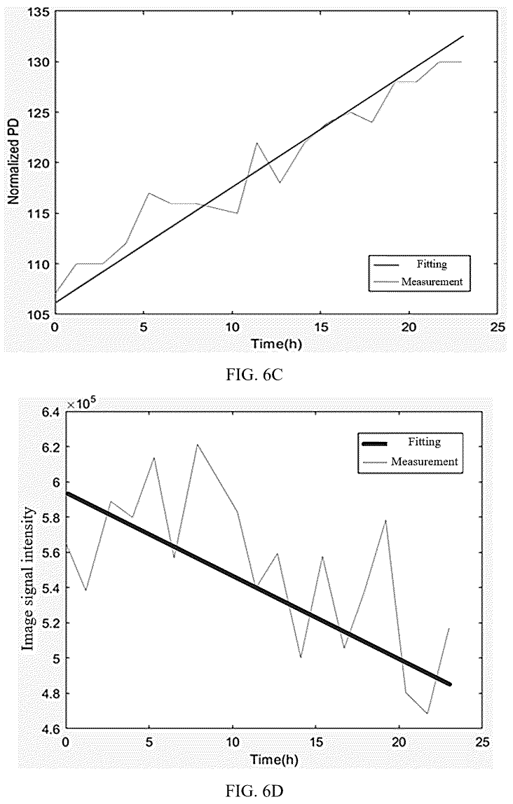

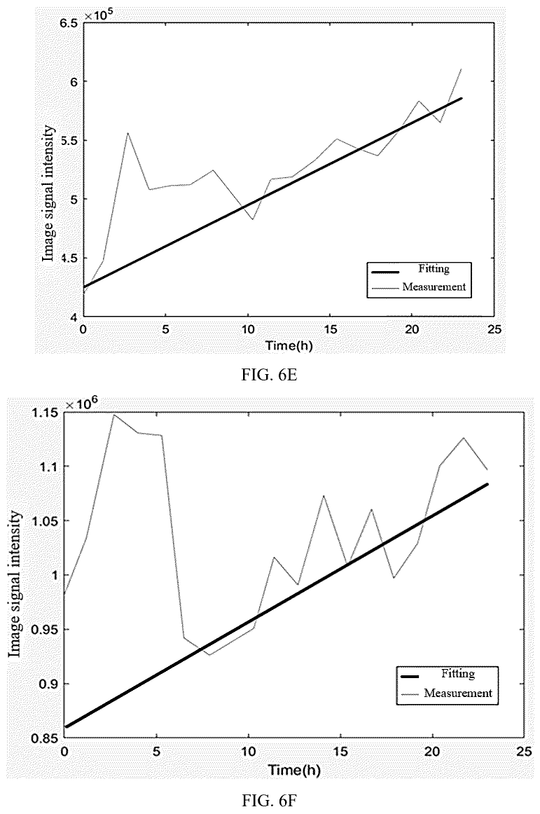

According to the above measurement method, the normalized values of T1, T2, and PD measured hourly are shown by broken lines in FIGS. 6A-6C. Since the data does not change stably over time, it is believed that a single-component model cannot explain the phenomenon of cerebral infarction at different onset times. In the literature, Sean C. L. Deoni and others have also proposed that there is a multi-component analysis model for the characteristics of tissues such as T1 in magnetic resonance scanning. Based on the results of this experiment and the physiological process of the cerebral infarction, it is believed that after the occurrence of the cerebral infarction, there are water molecules within the lesion that can be visualized by magnetic resonance imaging, which gradually change from the characteristics of cerebral parenchyma to those similar to edema (long T1, long T2), and the proportion of this change increases over time. Some of these water molecules may come from the transformation of water molecules in the cerebral parenchyma, and some may be from external water entering the lesion, which continues to increase over time. Therefore, it is believed that the physiological process of cerebral infarction tissue can be simulated using two components, namely cerebral parenchyma and edema-like components, with their proportions changing over time. Additionally, since the layer thickness collected in this experiment is 7 mm, and the pig's brain is relatively small, the thickness of the brain tissue slices at the medulla oblongata is less than 7 mm. The scanning slice at this location contains cerebrospinal fluid (CSF) signals. Therefore, the simulation calculation model for this experiment includes three different components, namely cerebral parenchyma, CSF, and edema-like components. Due to the entry of external water, the content of cerebral parenchyma decreases over time, reflected by a decrease in its PD value. The content of edema-like components increases over time, manifested by an increase in their PD value. The CSF, as a real existing tissue in the slice, remains unchanged in content.

Thus, the simulation formula described in the measurement method 1 (T1 measurement value of the tissue) is modified to a multi-component formula for the stroke experiment, as follows:
multi-component T1 acquisition sequence signal fitting formula:

$$S ( τ ) = {\sum }_{i = 1}^{N} P {D}_{i} ( 1 - 2 exp ( - \frac{τ}{T {1}_{i}} ) + exp ( - \frac{T R - T {E}_{last}}{T {1}_{i}} ) )$$

multi-component T2 acquisition sequence signal fitting formula:

$$S ( β ) = {\sum }_{i = 1}^{N} P {D}_{i} exp ( - \frac{β}{T {2}_{i}} )$$

signal fitting formula for multi-component T1WI imaging and FLAIR imaging:

$$S = {\sum }_{i = 1}^{N} P {D}_{i} ( 1 - 2 exp ( - \frac{T I}{T {1}_{i}} ) + exp ( - \frac{T R - T {E}_{last}}{T {1}_{i}} ) ) \times exp ( - \frac{T E}{T {2}_{i}} )$$

signal fitting formula for multi-component T2WI imaging and PD imaging:

$$S = {\sum }_{i = 1}^{N} P {D}_{i} ( 1 - exp ( - \frac{T R}{T {1}_{i}} ) ) \times exp ( - \frac{T E}{T {2}_{i}} )$$

where i=1, 2, . . . , N, N represents N types of components, according to the above description, N is 3 representing the cerebral infarction experiment. The meanings of T1, T2, and PD are the same as the above description, and a subscript i represents a value of an i-th type of tissue.

The expressions for the PD values of the three types of tissues changing over time are as follows:
a content of cerebral parenchyma decreases over time t (in hours), and an expression is:

$$P {D}_{cerebral parenchyma} = 1 \text{.15} - {b}_{c e r e b r a l p a r e n c h y m a} t$$

a content of CSF remains unchanged, and an expression is:

$${PD}_{CSF} = {PD}_{0 CSF}$$

a content of edema-like tissue increases over time t (in hours), and an expression is:

$$P {D}_{e dema - like tissue} = 0 + {b}_{edema - like tissue} t$$

the PD formulas of the three types of tissues are substituted into the first formulas for calculation.

T1 measurement acquisition sequence scanning parameters: In a first round of scanning, the repetition time TR is 4000 ms, and the last echo time TE is 107 ms. A series of TI values, denoted as τ, are set to [100 150 200 250 400 600 800 1000] ms. In a second round of scanning, the repetition time TR is 10000 ms, and the last echo time TE remains at 107 ms. The series of TI values, i.e., τ, are set to [3500 4000 4500] ms. A series of signal intensity values S(τ) corresponding to the TI values are obtained. By numerically fitting the data of τ and S(τ) using the first formula, the T1 measurement value is finally derived.

T2 measurement acquisition sequence scanning parameters: TR is 4000 ms, and a series of echo times β are set to [42, 56, 70, 84, 98, 112] ms. A series S(β) values corresponding to the echo times β are obtained. By numerically fitting the data of β and S(β) using the second formula, the T2 measurement value is finally derived.

T1WI scanning parameters: TR 1420 ms, TE 24.3 ms, TI 425 ms.

FLAIR image scanning parameters: TR 4380 ms, TE 104 ms, TI 1600 ms.

PD image scanning parameters: TR 10000 ms, TE 21 ms.

T2WI image scanning parameters: TR 2800 ms, TE 104 ms.

In this experiment, for the three types of components, the T1 values and the T2 values of cerebral parenchyma and CSF have been measured and are fixed as indicated by the above formulas. The unknown variables in the above formulas are the time variation rate b_{cerebral parenchyma} of cerebral parenchyma PD, the PD values of CSF, the T1 values and the T2 values of edema-like tissue, and the time variation rate b_{edema-like tissue} of edema-like tissue PD. Integrating these six sets of data and substituting them into their respective formulas, numerical fitting is performed to obtain the values listed in the following table:

TABLE 2

| | Brain | | Edema-like |
|---|---|---|---|
| | parenchyma | CSF | tissue |
| T1 (ms) | 420 | 4200 | 2200 |
| T2 (ms) | 105 | 2200 | 500 |
| PD | 1.15-0.00625 t | 0.15 | 0.00167 t |
| (normalization) | | | |

Note:
1. The unit of time t is hour.

2. Due to insufficient anesthesia in pigs at the 6th hour, which causes motion artifacts, the experimental data from the first 6 hours are not included in the fitting calculations. The subsequent values are highly correlated with the fitted values.

### 5. Parameters for Cerebral Hemorrhage Discrimination Sequence

Based on the data obtained from 3 and 4, the discrimination of cerebral hemorrhage uses an inversion recovery sequence (also referred to as inversion recovery pulse sequence), and its relative signal intensity formula is as follows:

$$S = {\sum }_{i = 1}^{N} P {D}_{i} ( 1 - 2 exp ( - \frac{T I}{T {1}_{i}} ) + exp ( - \frac{T R - T {E}_{last}}{T {1}_{i}} ) ) \times exp ( - \frac{T E}{T {2}_{i}} )$$

Using this formula for simulation calculations, considering in actual scanning to minimize the impact of T2 effects on the signal as much as possible, the TE of the scanning sequence adopts the system's minimum TE value, making

$$exp ( - \frac{T E}{T {2}_{i}} )$$

as close to 1 as possible, thus minimizing the T2 effect on the signal. The contrast between cerebral hemorrhage and cerebral infarct tissue and cerebral parenchyma is optimal. The longer the TE, the closer the contrast between cerebral hemorrhage and cerebral infarct and cerebral parenchyma, which is not conducive to differentiation, but it is also possible to appropriately select a TE slightly higher than the minimum TE value to distinguish the contrast between cerebral hemorrhage and cerebral infarct and cerebral parenchyma. After a certain TR value is given, TI and relative signal intensity S are numerically fitted according to the above formulas. For cerebral parenchyma and cerebral hemorrhage as a single-component model N=1, the T1, T2, PD values use the parameters in Table 1. For the cerebral ischemia model, since the cerebral infarct tissue does not contain cerebrospinal fluid, cerebrospinal fluid is excluded, and a two-component model is used, N=2, simulating two components of cerebral parenchyma and edema-like tissue. The T1, T2, PD values use the data in Table 2. The cerebral ischemia model simulates the signal changes every hour within 24 hours, and the PD values within 24 hours use the initial values and linear variation coefficients in Table 2.

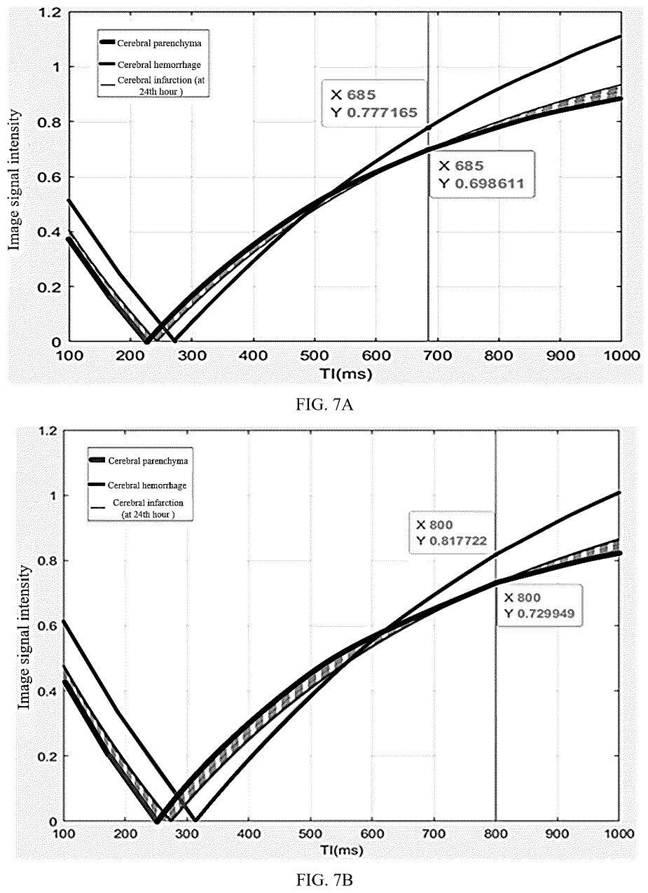

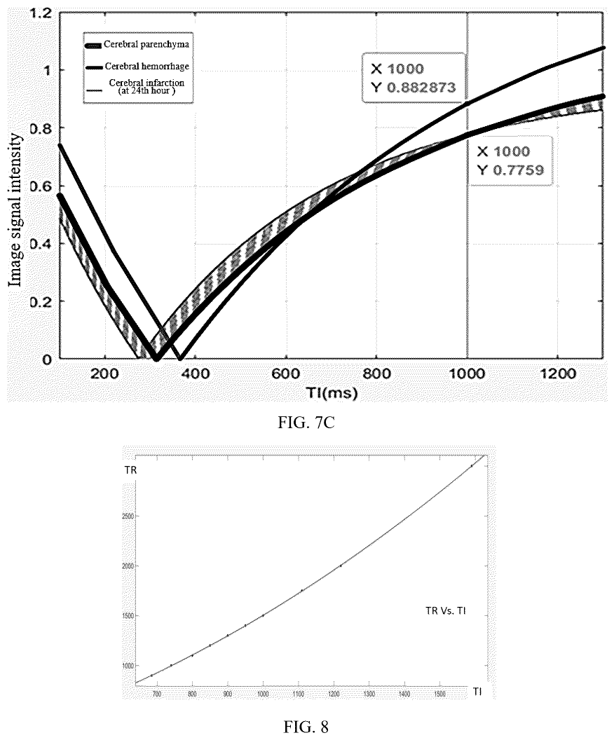

Through fitting calculations, it is found that for any given TR value, there exists a critical point for signal intensity. This critical point results in the cerebral parenchyma being isointense with the cerebral infarct tissue from 1 to 24 hours, while the hemorrhage signal is greater than the signals from the cerebral parenchyma and the cerebral infarct tissue over the 1 to 24 hours period. This signal intensity manifests on the image as bright signals for cerebral hemorrhage, and isointense signals for cerebral parenchyma and cerebral infarct tissue over 1 to 24 hours. FIGS. 7A-7C take TR values of 900 ms, 1100 ms, and 1500 ms as examples. The legends indicate that the solid lines with different thicknesses correspond to signals from cerebral hemorrhage, cerebral parenchyma, and cerebral infarct at the 24^{th} hour, while the dashed lines correspond to signals from the cerebral infarct tissue from the first to the 23rd hour after the onset of infarction. It can be concluded that the critical points are at TI 685 ms for TR 900 ms, TI 800 ms for TR 1100 ms, and TI 1000 ms for TR 1500 ms.

A series of TR values [900 1000 1100 1200 1300 1400 1500 1750 2000 3000 ms] are selected for simulation, and a corresponding series of critical points TI are obtained. These are fitted with a quadratic polynomial, as shown in FIG. 8. The optimal correspondence between TR and TI can be described by the following fitting results:

$$TR = 0.0006925 \times T {I}^{2} + 0.7426 \times T I + 67.78 .$$

Generally, when the corresponding TI value of is a fixed value, and the TR value fluctuates up and down by 10% according to the fitting result; or the TR value is a fixed value, and the corresponding TI value fluctuates up and down by 10% according to the fitting result, which has little impact on image quality.

The general principle for selecting combinations of TR values and corresponding TI values is to make the signal from cerebral hemorrhage appear as high signal, and the signals from cerebral infarct tissue and cerebral parenchyma appear as isointense or low signal. The selection of TR and TI should comply with the principle of the fastest clinical scanning speed. Additionally, the scanning time requirements can be appropriately relaxed within the patient's acceptable range to enhance the contrast between cerebral hemorrhage and cerebral infarct tissue and cerebral parenchyma, making them easier to distinguish.

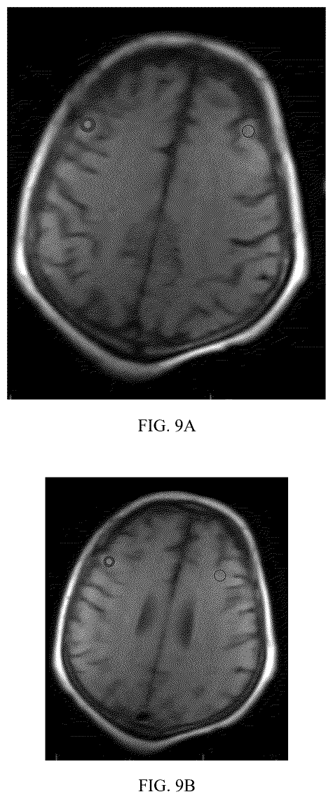

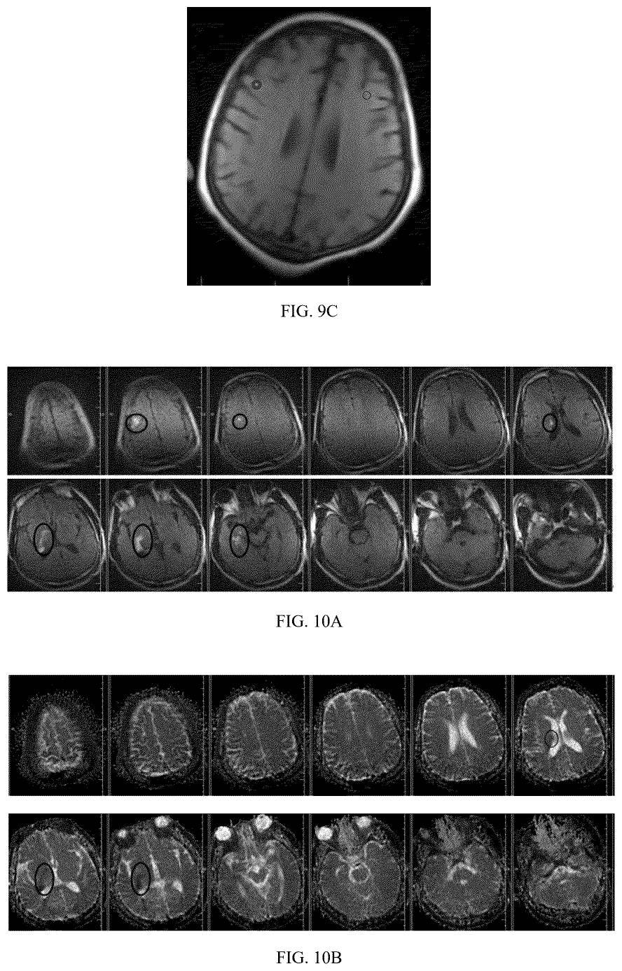

Clinical results selected TR=900 ms, 1100 ms, and 1500 ms for testing, and the results are shown in FIG. 9A-9C. The grayscale values of the hemorrhagic focus (marked in thick circle) and the contralateral normal brain tissue (marked in thin circle) are measured and compared with the simulation results. The results are as follows in the table:

TABLE 3

| | Experimental measurement | | | Simulation calculation | | |
|---|---|---|---|---|---|---|
| Testing | | Normal | | | Normal | |
| group | Hemorrhage | Brain | Ratio | Hemorrhage | Brain | Ratio |
| TR/TI (ms) | Brightness | Tissue | value | Brightness | Tissue | value |
| 900/685 | 1698 | 1533 | 110.7% | 0.777 | 0.699 | 111.2% |
| 1100/800 | 1575 | 1401 | 112.4% | 0.818 | 0.730 | 112.1% |
| 1500/1000 | 1642 | 1447 | 113.4% | 0.883 | 0.776 | 113.8% |

It can be seen that the experimental results are highly consistent with the fitted values.

### 6. Stroke Case

The following clinical trial is conducted using equipment with a low-field magnetic resonance stroke differentiation sequence to verify its ability to quickly and accurately determine hemorrhagic stroke. The equipment utilized is the mobile head and neck magnetic resonance system ACUTA Elfin, manufactured by Foshan Ruijatu Medical Technology Co., Ltd. The TR/TI combination used in the case described below is 1100/800 ms, and the TE is the minimum value of the system, which is 24 ms.

First case: A 47-year-old male patient initially presents with weakness in the left limbs for 24 hours. After being admitted to the hospital, a CT scan confirms a diagnosis of cerebral infarction. The male patient then undergoes scanning using the mobile head and neck MRI system ACUTA Elfin, and the images obtained are shown in FIGS. 10A-10D. The DWI images reveal multiple bright signals in the right frontal lobe and basal ganglia areas. The ADC map shows low signals in the corresponding lesion areas, while the frontal lobe appears isointense. The FLAIR images display high signals in the corresponding lesion areas, and the PD images show isointense signals in the lesion areas. Based on these images and in conjunction with a comprehensive clinical assessment, this case is determined to be an acute/subacute phase of multiple cerebral infarctions.

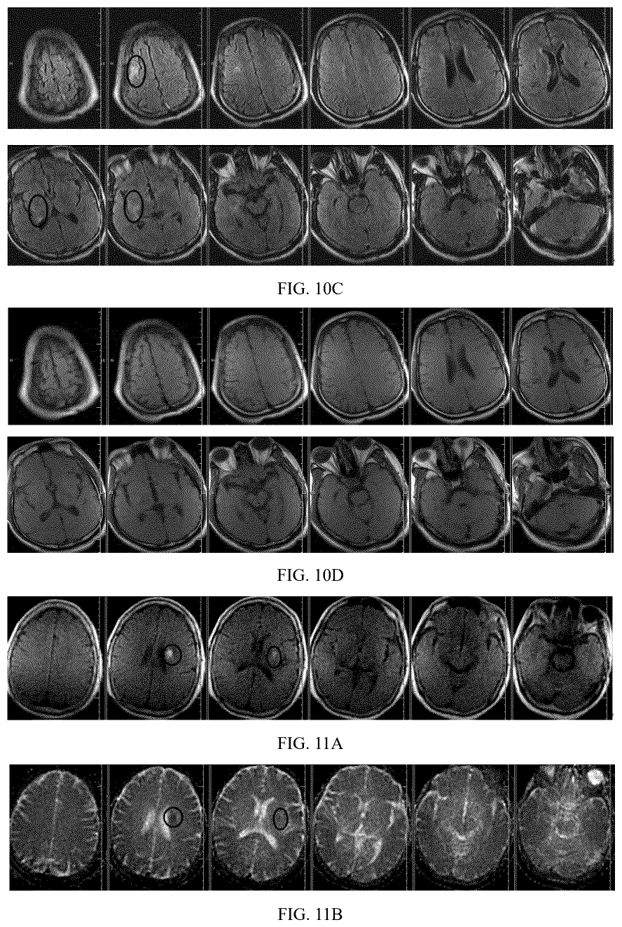

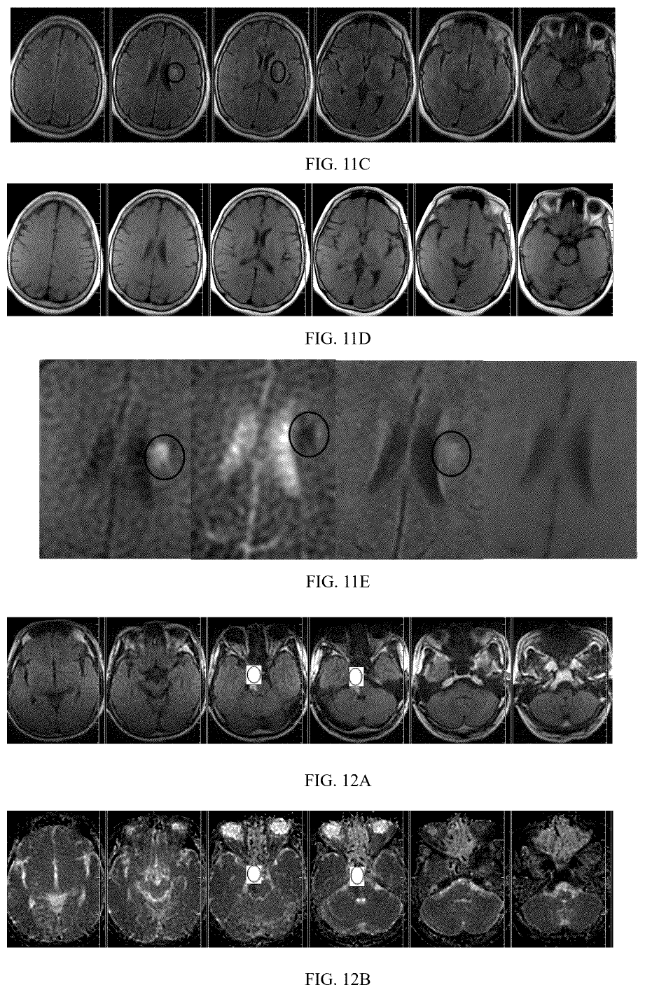

Second case: A 64-year-old female patient presents with slurred speech and general weakness for 24.6 hours. After admission, she undergoes scanning using the mobile head and neck MRI system ACUTA Elfin, and the images obtained are shown in FIGS. 11A-11E. The DWI images show a bright signal along the upper part of the left basal ganglia, with the corresponding lesion area on the ADC map displaying a low signal. The FLAIR images exhibit a high signal in the corresponding lesion area, and the PD images show an isointense signal in the lesion area. Based on these images and in conjunction with a comprehensive clinical assessment, this case is determined to be an acute cerebral infarction. In contrast, the CT scan suggests the possibility of cerebral infarction but recommends further examination and cannot provide an accurate diagnosis.

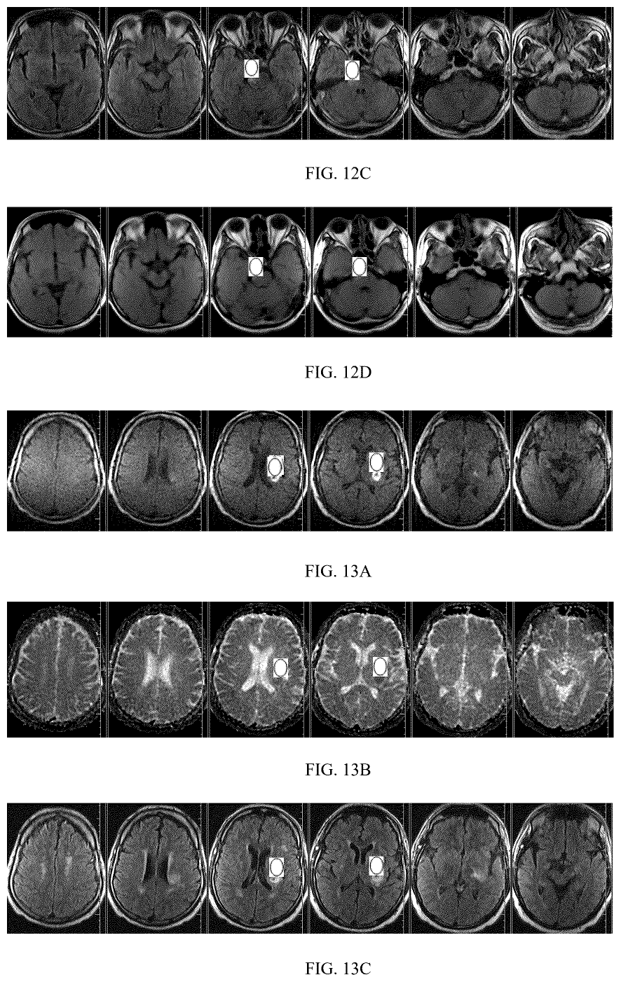

Third case: A 50-year-old male patient experiences dizziness and sudden weakness in the left limbs for 29 hours. After being admitted to the hospital, he is scanned using the mobile head and neck MRI system ACUTA Elfin, and the resulting images are shown in FIGS. 12A-12D. The DWI images reveal a bright signal in the right brainstem, with the corresponding lesion area on the ADC map showing a low signal. The FLAIR images indicate a high signal in the corresponding lesion area. An initial judgment suggests a stroke. The PD images show a bright signal in the corresponding lesion area. Based on these images and in conjunction with a comprehensive clinical assessment, this case is determined to be an acute cerebral hemorrhage. Additionally, the CT report indicates an acute cerebral hemorrhage, which is consistent with the imaging results from the mobile head and neck MRI system ACUTA Elfin.

Fourth case: A 50-year-old male patient is initially diagnosed with numbness on the right side of his body. Upon admission to the hospital, he undergoes scanning using the mobile head and neck MRI system ACUTA Elfin, resulting in the images shown in FIGS. 13A-13D. The DWI images display bright signals in the left basal ganglia and corona radiata areas, with the corresponding lesion area on the ADC map showing a low signal. The FLAIR images indicate high signals in the corresponding lesion area, leading to a preliminary judgment of a stroke. The PD images show a bright signal in the corresponding lesion area. Based on these images and in conjunction with a comprehensive clinical assessment, this case is determined to be an acute cerebral hemorrhage. Additionally, the CT report indicates an acute cerebral hemorrhage, which is consistent with the imaging results from the mobile head and neck MRI system ACUTA Elfin.

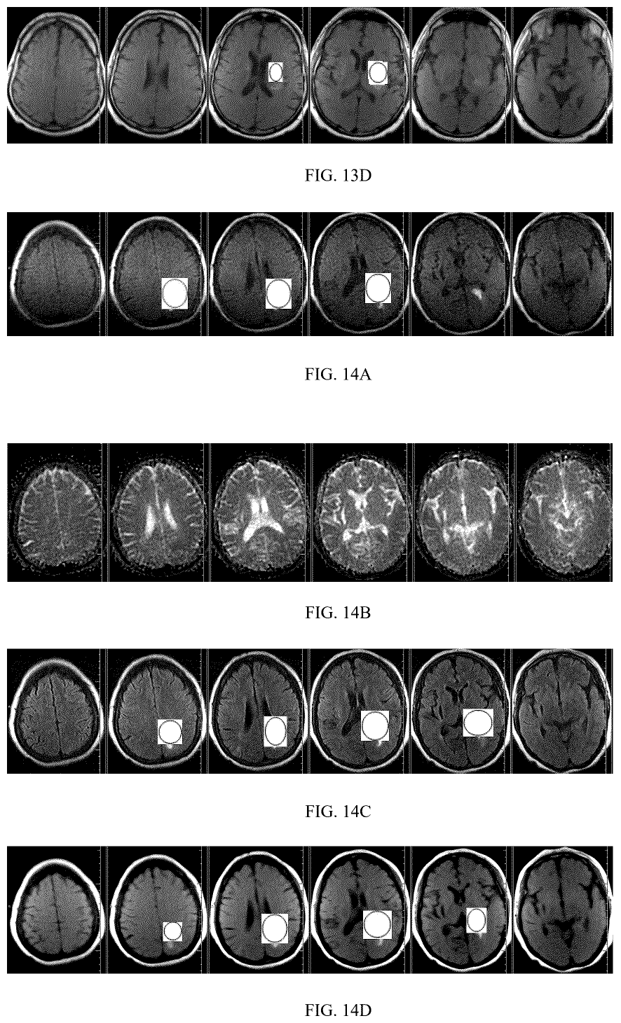

Fifth case: A 59-year-old female patient is initially diagnosed with visual disturbances and headache for 24 hours upon admission. She then undergoes scanning using the mobile head and neck MRI system ACUTA Elfin, resulting in the images shown in FIGS. 14A-14D. The DWI images reveal a bright signal in the left posterior ventricle area, with the corresponding lesion area on the ADC map showing a low signal. The FLAIR images indicate a high signal in the corresponding lesion area, leading to a preliminary judgment of a stroke. The PD images show a bright signal in the corresponding lesion area. Based on these images and in conjunction with a comprehensive clinical assessment, this case is determined to be a subacute cerebral hemorrhage. Additionally, the CT report was inconclusive, but subsequent clinical validation confirmed that this case is indeed a subacute cerebral hemorrhage.

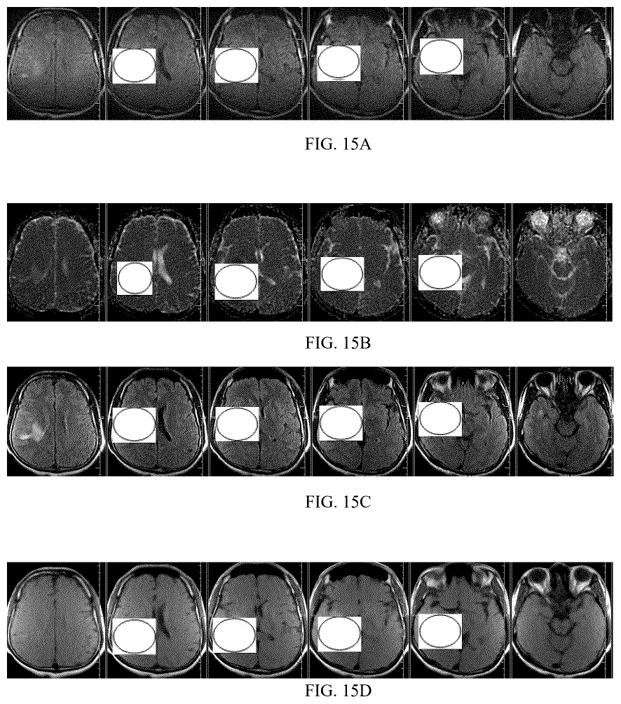

Sixth case: A 43-year-old male patient is initially diagnosed with sudden weakness in the left limbs without any apparent cause 5.5 hours prior to admission. He then undergoes scanning using the mobile head and neck MRI system ACUTA Elfin, resulting in the images shown in FIGS. 15A-15D. The DWI images show extensive bright signals in the right basal ganglia, insular cortex, and corona radiata regions, with the corresponding lesion area on the ADC map displaying a low signal. The FLAIR images indicate high signals in the corresponding lesion area, leading to a preliminary judgment of a stroke. The PD images reveal a bright signal in the corresponding lesion area. Based on these images and in conjunction with a comprehensive clinical assessment, this case is determined to be an acute cerebral hemorrhage. Additionally, the CT report indicates an acute cerebral hemorrhage, which is consistent with the imaging results from the mobile head and neck MRI system ACUTA Elfin.

What is claimed is:

1. A low field magnetic resonance imaging (MRI) stroke identification sequence, using an inversion recovery sequence, wherein a formula of a relative signal intensity of the inversion recovery sequence is expressed as follows:
$$S = {\sum }_{i = 1}^{N} P {D}_{i} ( 1 - 2 exp ( - \frac{T I}{T {1}_{i}} ) + exp ( - \frac{T R - T {E}_{last}}{T {1}_{i}} ) ) \times exp ( - \frac{T E}{T {2}_{i}} )$$
where S represents the relative signal intensity; PD represents a proton density of tissue; TI represents an inversion recovery time of the inversion recovery sequence; T1 represents a T1 relaxation time of the tissue; TR represents a repetition time of the inversion recovery sequence; TEₗₐₛₜ represents a last echo time in a multi-echo sequence; TE represents an effective echo time; T2 represents a T2 relaxation time of the tissue; i=1, 2, . . . , N, N represents N types of components, cerebral parenchyma and cerebral hemorrhage are modeled as a single-component model with N=1, cerebral infarction tissue is modeled as a two-component model with N=2, and a subscript i represents a value of an i-th type of tissue;
wherein the TE is set to a minimum value or a near-minimum value achievable by a system, thereby making
$$exp ( - \frac{T E}{T {2}_{i}} )$$
as close to 1 as possible, and minimizing an influence of a T2 effect on the relative signal intensity S; and
wherein a combination of a TR value and a corresponding TI value is selected, thereby making a signal from the cerebral hemorrhage as a high signal, and making a signal from the cerebral infarction tissue and the cerebral parenchyma appear as an isointense signal or a low signal.

2. The low field MRI stroke identification sequence as claimed in claim 1, wherein selection of the combination of the TR value and the corresponding TI value conforms to a principle of a fastest clinical scanning speed.

3. The low field MRI stroke identification sequence as claimed in claim 1, wherein a corresponding relationship between the TR value and the corresponding TI value conforms to a fitting result as follows:
$$TR = 0.0006925 \times T {I}^{2} + 0.7426 \times T I + 67.78 .$$

4. The low field MRI stroke identification sequence as claimed in claim 3, wherein the corresponding TI value is a fixed value, and the TR value fluctuates up and down by 10% according to the fitting result; or the TR value is a fixed value, and the corresponding TI value fluctuates up and down by 10% according to the fitting result.

5. The low field MRI stroke identification sequence as claimed in claim 1, wherein a strength of the low field is 0.23 Tesla (T), a TE value is 24 milliseconds (ms), the TR value is 900 ms, and the corresponding TI value is 685 ms.

6. The low field MRI stroke identification sequence as claimed in claim 1, wherein a strength of the low field is 0.23 T, a TE value is 24 ms, the TR value is 1100 ms, and the corresponding TI value is 800 ms.

7. The low field MRI stroke identification sequence as claimed in claim 1, wherein a strength of the low field is 0.23 T, a TE value is 24 ms, the TR value is 1500 ms, and the corresponding TI value is 1000 ms.

8. The low field MRI stroke identification sequence as claimed in claim 1, wherein a PD value is obtained by measuring a signal intensity of a proton weighted image.

9. The low field MRI stroke identification sequence as claimed in claim 1, wherein inversion recovery pulses with a series of different TI values are applied to the relative signal intensity formula before using a fast spin echo sequence, thereby making a signal magnitude of a region of interest (ROI) being related to a T1 value; and a T1 measurement value is obtained by fitting using a formula expressed as follows:
$$S ( τ ) = α \times ( 1 - 2 exp ( - \frac{τ}{T 1} ) + exp ( - \frac{T R - T {E}_{last}}{T 1} ) )$$
where S(τ) represents a signal intensity of the tissue; α represents a weight coefficient; and τ represents a series of different TI values.

10. The low field MRI stroke identification sequence as claimed in claim 1, wherein other parameters constant except for the TE are kept unchanged to obtain a T2 measurement value based on a correlation between the TE and the T2 by using a fast spin echo sequence; and the T2 measurement value is obtained by fitting using a formula expressed as follows:
$$S ( β ) = α \times exp ( - \frac{β}{T 2} )$$
where S(β) represents a signal intensity of the tissue; α represents a weight coefficient; and β represents a series of echo times.

11. A device for identifying the low field MRI stroke identification sequence, comprising a magnetic resonance scanner, wherein the magnetic resonance scanner is configured to identify the low field MRI stroke identification sequence as claimed in claim 1.
